(19) United States
(12) Patent Application Publication
(10) Pub. No.: US 2008/0202530 A1
(43) Pub. Date: Aug. 28, 2008

(54) Device and Method for the Treatment of Movement Disorders

(76) Inventors: Anthony B. Sims, Columbia, MD (US); Brendan Stack, Vienna, VA (US)

Correspondence Address: CAHN & SAMUELS LLP, 1100 17th STREET NW, SUITE 401, WASHINGTON, DC, 20036, US

(21) Appl. No.: 12/031,614

(22) Filed: Feb. 14, 2008

Related U.S. Application Data
(60) Provisional application No. 60/890,156, filed on Feb. 15, 2007.

(51) Int. Cl. A61F 5/01; A61C 19/00

(52) U.S. Cl. 128/845; 433/140; 29/896.1

(57) ABSTRACT

An orthotic device and method that reduces or eliminates the physical effects associated with movement disorders, such as Tourette's Syndrome, and temporomandibular joint (TMJ) disorders. Specifically, the device and method eliminates or substantially reduces the involuntary movements and premonitory urges associated with movement disorders without the need for medication or surgery. The device and method also eliminates or substantially reduces the pain, nerve stimulation and other effects associated with TMJ disorders, such as displaced articulated disks. The device includes a base portion and adjustable raised spacers that adjust the position of the mandible to relieve pressure exerted on the trigeminal nerve (the fifth cranial nerve or CN V), retrodiscal tissue, and other nerves due to improper TMJ alignment. The liberation of pressure and stimulation on the trigeminal nerve, retrodiscal tissue and other nerves eliminates the involuntary movements, pain and stimulation associated with these conditions. The method also relieves other associated conditions, such as headaches or migraines, sleep disorder breathing, vertigo, and the like.

This patent application claims the benefit of U.S. Provisional Patent Application No. 60/890,156, filed Feb. 15, 2007, which is hereby incorporated by reference.

### I. FIELD OF THE INVENTION

The present invention is related to a device and method for treating the effects of movement disorders, such as Tourette's Syndrome, and temporomandibular joint (TMJ) disorders. More particularly, the present invention relates to a dental orthotic device and method for reducing or eliminating the tics and involuntary movements associated with movement disorders without the need for medication or surgery.

### II. BACKGROUND OF THE INVENTION

It has been estimated that approximately 6 million people in the United States suffer from some type of movement disorder. Movement disorders affect the quality, speed, fluidity and ease of movement. Movement disorders manifest in a variety of physical conditions, including tics (involuntary muscle contractions that often result in blinking, coughing, sniffing, shoulder shrugging, neck stretching, throat clearing, facial twitching, grunting, sighing, etc.), tremors (involuntary shaking or quivering), dystonia (causes involuntary movement and prolonged muscle contraction), ataxia (lack of coordination, often producing jerky movements), myoclonus (abrupt, irregular muscle spasms or twitches), as well as other conditions that cause involuntary tics, tremors and body movements. Waxing and waning motor tics are often accompanied by premonitory urges.

Movement disorders and their physical manifestations are also associated with and accompany chronic disorders such as Tourette's syndrome, Parkinson's disease, Huntington's disease, etc. Further, movement disorders are often accompanied by headaches or migraines, sleep disorder breathing, temporomandibular joint (TMJ) disorder or jaw pain, vertigo, and a host of other related ailments.

There also exists a group of disorders that are collectively referred to as thoracic outlet syndrome (TOS). TOS consists of a group of distinct disorders affecting the brachial plexus (nerves that pass from the neck into the arms) and various nerves and blood vessels between the base of the neck and axilla (armpit). This group includes true neurologic, disputed, arterial, and traumatic disorders. These disorders are complex, poorly defined, and are generally unrelated except that they each exhibit symptoms affecting the upper limbs. These associated symptoms include pain, weakness, fatigue, numbness, muscle wasting, sensory loss, burning sensations, and other similar effects.

Tourette's syndrome is perhaps the most well-known movement disorder. Tourette's syndrome is marked by multiple motor and vocal tics. Symptoms associated with Tourette's usually manifest during childhood or early adolescence and vary in frequency and severity. Individuals who suffer from Tourette's syndrome are often diagnosed to have various behavioral problems such as attention deficit hyperactive disorder (ADHD), sleep disorders, obsessive compulsive disorder (OCD), and impulsivity. For the most part, the causes of Tourette's syndrome have been misunderstood. It has been generally believed that these disorders were caused by genetic, psychological or neurological conditions. However, the exact causes have largely remained unidentified.

Movement disorders have profound effects on the lives of those who suffer from the diseases. Depending on the severity, these individuals often find it difficult to assimilate socially and are frequently socially ostracized. Treatment of movement disorders, such as Tourette's syndrome, and the multiple comorbid disorders includes medicines and surgery. However, these treatments are unreliable and have many side effects, such as severe fatigue and drowsiness which are often more socially and professionally detrimental than the effects of the disorder. In the case of surgery, which can include brain surgery, the cost can be prohibitive and the risk for permanent injury and diminished capacity can be extreme.

Movement disorders and their associated affects have been connected to temporomandibular joint (TMJ) disorders, such as misalignment of the TMJ, displacement of the articulated disk of the TMJ, impingement of the trigeminal nerve or fifth cranial nerve (CN V) by a displaced TMJ, and related maladies. As illustrated in FIG. 1, the trigeminal nerve (CN V) 100 is the largest cranial nerve and has three divisions: the ophthalmic division 110, the maxillary division 120, and the mandibular division 130. The maxillary division 120 and mandibular division 130 are both sensory with the mandibular division 130 having both sensory and motor functions.

The pathways of the CN V 100 are such that impulses travel from proprioceptive receptors, via the A alpha and C nerve fibers to the Gasserian ganglion to the level of C1 or C2. Then the spinal tract of V turns caudally at the level of C1 or C2. It is at this level where the neurons of the subnucleus caudalis cross over within the spinal cord and second order neuron within the spinal cord (substantia gelatinosa) travel upward towards the thalamus and then onto the cerebral cortex. This condition results in the stimulation of many other CN nerves, including for example the VII, IX, X, and other neural elements that are intimately involved with movement disorders and cause reflex reactions with the nerves.

Humans have two TMJs, one on either side of the cranium or skull, that function by working in unison. Each TMJ is formed of two bones: the upper temporal bone that forms part of the skull, and the lower jaw bone known as the mandible. Hence the term temporomandibular joint (TMJ). As illustrated in FIGS. 2A-2E, the two bones that form the TMJ 200, the temporal bone 210 and the mandible 220, are separated by an articulated disk 230 that forms part of a synovial joint. The articulated disk 230 is a thin, oval-shaped piece of fibrocartilage (flexible and resilient cartilage) of varying thickness located between the temporal bone 210 and the mandible 220 that helps to ensure the proper function of the TMJ 200. The articulated disk 230 partitions the synovial joint into an upper (superior) compartment and a lower (inferior) compartment. The upper and lower compartments are each formed with the articulated disk 230 and the temporal bone 210 and mandible 220 and allow for the translational movement of the lower jaw and rotational movement, respectively. The translational movements involve the sliding forward or side-to-side of the lower jaw. The rotational movements involve opening and closing of the jaw. The articulated disk 230 also includes a posterior portion called the retrodiscal tissue 235. The retrodiscal tissue 235 is highly vascular and innervated connective tissue containing a matrix of blood vessels and nerves, particularly the auriculotemporal nerve or mandibular branch of the trigeminal (CN V) nerve.

FIGS. 2A and 2B illustrate exemplary depictions of a normally functioning TMJ. As illustrated in FIG. 2A, when the mouth is closed the rounded end or condyle 225 of the mandible 220 rests adjacent to the fossa 215 of the temporal bone 210. In a normal or properly aligned TMJ, the condyle 225 and fossa 215 are connected to and separated by the articulated disk 230. Retrodiscal tissue 235 rests in a posterior position along the base of the temporal bone 210. As the mouth begins to open, the condyle 225 of the mandible 220 rotates in the lower compartment of the articulated disk 230. As the mouth continues to open, as illustrated in FIG. 2B, the mandible 220 and the entire articulated disk 230 moves forward along the temporal bone 210 as the condyle 225 rotates in the lower compartment. The retrodiscal tissue 235 extends along the base of the temporal bone 210 out of contact with the mandible 220.

The normal spacing between the condyle 225 of the mandible 220 and the fossa 215 of the temporal bone 210 should be approximately 3 millimeters (mm) in order to support the articulated disk 230 between the two. However, if this spacing is insufficient to support the articulated disk 230 or the space is reduced or restricted and the condyle 225 pinches the retrodiscal tissue 135. Often the pain associated with TMJ 200 is a result of the mandible 220 contacting the retrodiscal tissue 235 due to a displaced or misaligned articulated disk. Further, in the case of Tourette's syndrome, the impingement or contact of the retrodiscal tissue does not provide enough stimuli to be painful. However, the constant neuritis or stimulation of the auriculotemporal nerve causes a lower level chronic irritation that triggers a constant input into the spinal nucleus of the CN V. This condition has also been shown to result in the stimulation of many other CN nerves, including for example the VII, IX, X, and other neural elements that are intimately involved with movement disorders and cause reflex reactions with the nerves, as discussed above.

FIGS. 2C and 2D illustrate exemplary depictions of a displaced or dislocated disk in the TMJ 200. In a closed mouth, as illustrated in FIG. 2C, the articulated disk 230 is displaced from the normal position between the condyle 225 of the mandible 220 and the fossa 215 of the temporal bone 210. The displaced articulated disk 230 leaves the retrodiscal tissue 235 exposed to contact by the condyle 225 which can cause severe and debilitating pain. FIG. 2D illustrates a displaced articulated disk 230 in an open mouth. As the mouth opens, the condyle 225 rotates and the mandible 220 moves forward. The displaced articulated disk 230 is pushed forward by the condyle 225 and the retrodiscal tissue 235 is extended along the base of the temporal bone 210. The displaced articulated disk 230 again leaves the retrodiscal tissue 235 exposed to contact by the condyle 225. As noted above this contact can lead to severe pain as well as effects associated with neuritis of the nerves.

Therefore, a need still exists for a more cost effective, low risk treatment of movement disorders and TMJ disorders that is reliable and does not require surgery or medication.

### III. SUMMARY OF THE INVENTION

In at least one exemplary embodiment, the present invention provides an apparatus for changing the alignment of the temporomandibular joint, including an orthotic base that affixes to the interior of a mouth; at least one raised platform attached to said base; and at least one adjustable spacer disposed inside said raised platform, wherein said adjustable spacer changes the height of said raised platform.

In at least one exemplary embodiment, the present invention provides an apparatus for changing the alignment of the temporomandibular joint, including an orthotic base that affixes to the interior of a mouth; at least one raised platform attached to said base, wherein said at least one raised platform adjusts the position of the mandible; and at least one adjustable spacer disposed inside said raised platform, wherein said adjustable spacer changes the height of said raised platform, said adjustable spacer including a substantially flat lower platform; a substantially flat upper platform positioned above said lower platform; at least one adjustable connection mechanism in communication with said lower bar and said upper bar, wherein said connection mechanism adjusts the position of said upper platform with respect to said lower platform thereby adjusting the height of said raised platform.

In at least one exemplary embodiment, the present invention provides a method of making a device for changing the alignment of the temporomandibular joint, including forming an orthotic device base, wherein said base is formed to affix to the interior of a mouth; attaching at least one raised platform to said base, wherein said at least one raised platform is designed to adjust the position of the mandible; and disposing at least one adjustable spacer inside said raised platform, wherein said adjustable spacer changes the height of said raised platform.

In at least one exemplary embodiment, the present invention provides a method for adjusting the position of the mandible, including taking an impression of a patient's mouth to be adjusted; determining an appropriate separation of the upper and lower jaw of said mouth; making an orthotic device having a base and adjustable raised portions, wherein said device integrates said mouth impression and said determined appropriate jaw separation; attaching said orthotic device to said mouth such that the raised portions are disposed adjacent the patient's molar teeth; adjusting the height of said raised portions to achieve the desired alignment of the mandible.

In at least one exemplary embodiment, the present invention provides the advantage of adjusting the position of the mandible in order to relieve pressure exerted on nerves and tissue proximate to the temporomandibular joint.

In at least one exemplary embodiment, the present invention provides the advantage of relieving pressure exerted on nerves and tissue proximate to the mandible in order to reduce or eliminate involuntary movements associated with movement disorders.

In at least one exemplary embodiment, the present invention provides the advantage of relieving pressure exerted on nerves and tissue proximate to said mandible in order to reduce or eliminate at least one additional condition associated with movement disorders.

In at least one exemplary embodiment, the present invention provides the advantage of adjusting or realigning the position of the mandible in order to properly position the articulated disk in the temporomandibular joint.

In at least one exemplary embodiment, the present invention provides the advantage of allowing for the proper positioning of the articulated disk within the temporomandibular joint in order to reduce or eliminate pain associated with a displaced temporomandibular joint.

In at least one exemplary embodiment, the present invention provides the advantage of allowing for the proper positioning of the articulated disk within the temporomandibular joint in order to reduce or eliminate at least one additional condition associated with a displaced temporomandibular joint.

In at least one exemplary embodiment, the present invention provides the advantage of adjusting the position of a raised platform in order to more precisely change the position of said mandible.

In at least one exemplary embodiment, the present invention provides the advantage of precisely changing the position of said mandible in order to allow the articulated disk to be properly positioned in said temporomandibular joint.

### IV. BRIEF DESCRIPTION OF THE DRAWINGS

The present invention is described with reference to the accompanying drawings, wherein:

FIG. 1 illustrates a human facial anatomy including trigeminal nerve (CN V) and associated cranial nerve divisions, including ophthalmic, maxillary, and mandibular.

FIG. 2A illustrates an exemplary cranial anatomy including the temporal bone and mandible the form the temporomandibular joint (TMJ).

FIG. 2B illustrates an exemplary internal anatomy of a normal temporomandibular joint (TMJ) with the mouth in a closed position.

FIG. 2C illustrates an exemplary internal anatomy of a normal temporomandibular joint (TMJ) with the mouth in an open position.

FIG. 2D illustrates an exemplary internal anatomy of a temporomandibular joint (TMJ) having a displaced articulated disk with the mouth in a closed position.

FIG. 2E illustrates an exemplary internal anatomy of a temporomandibular joint (TMJ) having a displaced articulated disk with the mouth in an open position.

FIG. 3 illustrates a perspective view of an orthotic device in accordance with an exemplary embodiment of the present invention.

FIG. 4 illustrates an orthotic device attached to the mouth in accordance with an exemplary embodiment of the present invention.

FIG. 5 illustrates a bottom view of an orthotic device in accordance with an exemplary embodiment of the present invention.

FIG. 6A illustrates a side view of a vertically adjustable spacer of the orthotic device in accordance with an exemplary embodiment of the present invention.

FIG. 6B illustrates a bottom view of a vertically adjustable spacer of the orthotic device in accordance with an exemplary embodiment of the present invention.

FIG. 7 illustrates a cutaway side view at lines X-X of the orthotic device illustrated in FIG. 3 in accordance with an exemplary embodiment of the present invention.

FIG. 8 illustrates a rear perspective view of an orthotic device in accordance with an exemplary embodiment of the present invention.

FIG. 9 illustrates the process of adjusting the temporomandibular joint (TMJ) in accordance with an exemplary embodiment of the present invention.

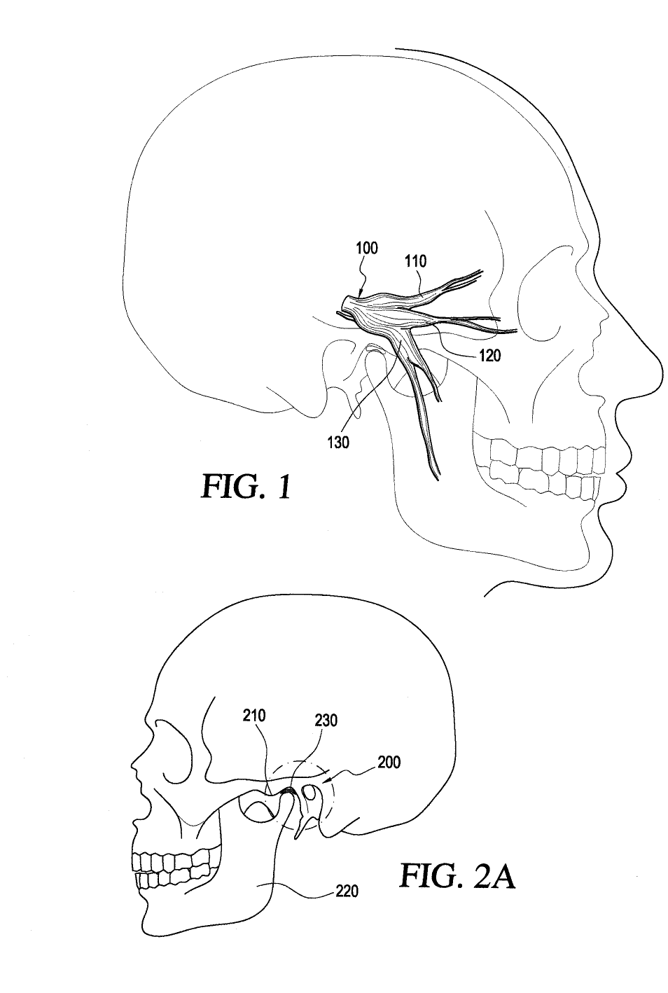

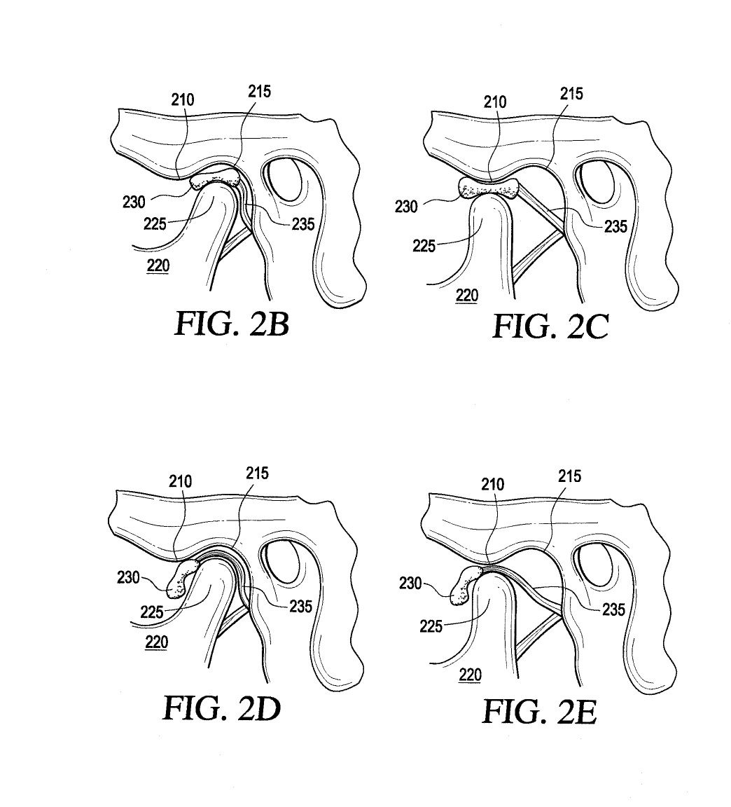

Given the following enabling description of the drawings, the apparatus and method should become evident to a person of ordinary skill in the art.

### V. DETAILED DESCRIPTION OF THE DRAWINGS

The present invention discloses an apparatus and method effective in eliminating or greatly reducing the source and symptoms associated with movement disorders, particularly Tourette's syndrome, including the involuntary movements and premonitory urges associated with these disorders. The present invention is also effective in eliminating or greatly reducing the source and symptoms associated with TMJ disorders, such as misaligned TMJs and displaced articulated disks. The apparatus of the present invention is an oral orthotic or orthopedic dental appliance (orthotic device) that is inserted into the mouth in order to affect the relative position of the components of the TMJ. The orthotic device allows the components of the TMJ to be shifted or adjusted thereby correcting or improving misalignments of the TMJ, and stresses and impingements of the various nerves proximate the TMJ thereby relieving the associated symptoms.

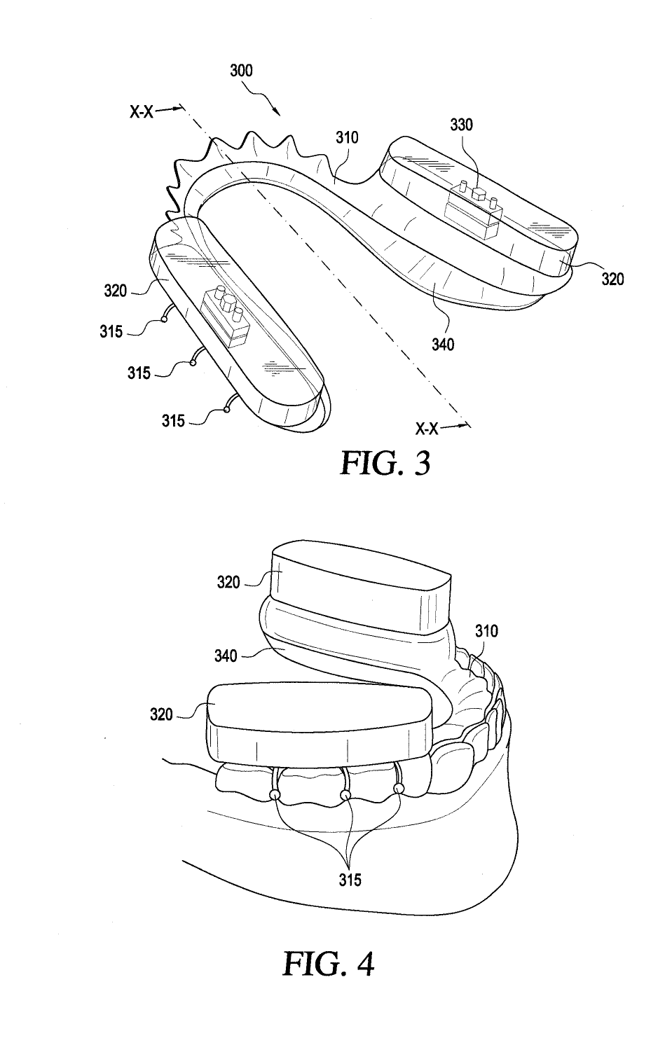

The present invention as illustrated, for example, in FIG. 3 is directed to an orthotic device 300 for the treatment of movement disorders. The orthotic device 300 preferably includes at least one base portion 310 that attaches to the upper or lower teeth (preferably lower) via connectors 315, such as Adams clasps or ball clasps, as shown in FIG. 4. The base portion 310 also includes raised platforms 320 on both sides of the base portion 310 and optional tongue retaining flanges 340. Raised platforms 320 are preferably located near the rear of the jaw adjacent the molar teeth and include vertically adjustable spacers 330 that alter the height of the raised platforms 320. The raised platforms 320 are vertically adjustable with respect to the base portion 310 and are sized as appropriate to adjust the position of mandible of a patient when in use. In alternative embodiments, base portion 310 may be limited to a single raised platform 320.

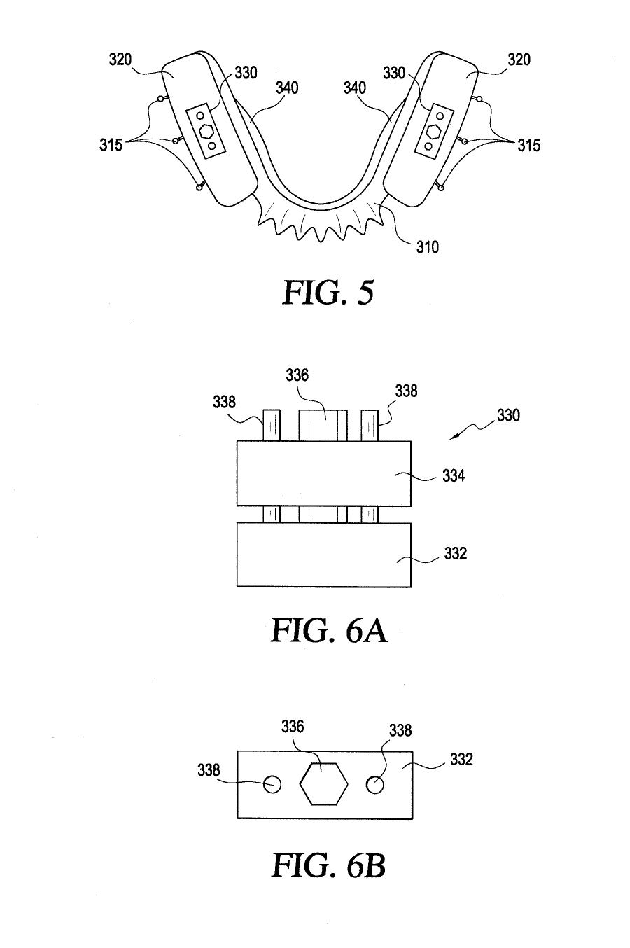

FIG. 5 illustrates a bottom view of the device of the present invention. This view shows the bottom of the base portion 310, including connectors 315, tongue retaining flanges 340, raised platforms 320 and vertically adjustable spacers 330 located in the raised platforms 320. The vertically adjustable spacers 330 are accessible via the bottom of the orthotic device 310.

FIGS. 6A and 6B, illustrate side and bottom views of the vertically adjustable spacers 330, respectively. FIG. 6A shows a vertically adjustable spacer 330, including a lower spacer platform 332, an upper spacer platform 334, an adjustment screw 336, such as an Allen or hexagonal screw, and guide rods 338. FIG. 6B shows the bottom of a vertically adjustable spacer 330, including the lower spacer platform 332, screw 336 and guide rods 338. In use, the upper spacer platform 334 is adjusted with respect to the lower spacer platform 332, i.e., up or down, by turning the adjustment screw 336 which is accessible via the bottom of the base portion 310. While the vertically adjustable spacer 330 has been described in terms of having two platforms, i.e. lower spacer platform 332 and upper platform 334, the vertically adjustable spacer 330 may also be designed with only one platform that adjusts to alter the position of the raised portion 320.

The vertically adjustable spacers 320 allow the height of the raised platforms 320 to be adjusted and tuned in order to more flexibly manipulate the position of the mandible and thereby relieve the effects associated with improper TMJ spacing, displaced articulated disks, and/or impinged nerves or retrodiscal tissue adjacent the TMJ. While the vertically adjustable spacers have been described in terms of particular embodiments, similar mechanisms that would be accessible from the exterior of the device to controllably adjust the height of the raised platforms would be suitable and within the scope of the present invention.

The orthotic device 300 is made of a resilient, non-toxic material and is designed and fitted to attach to the teeth of a patient. The base portion 310 is designed by making an impression of the patient's teeth. An appropriate height of the raised platforms 320 is determined by manually adjusting the spacing of the patient's upper and lower jaw. The spacing may be adjusted, for example, by stacking thin spacers on top of each other until an appropriate spacing or raised platform is determined. Once the appropriate height is determined, the orthotic device 300 is formed by integrating the patient's teeth impression and the determined platform spacing in order to form the base portion 310 and the raised platforms 320. The raised platforms 320 include vertically adjustable spacers 330 disposed inside the raised platforms 320. The base portion 310 and the raised portion 320 may be made of similar materials. However, while the raised platforms 320 are formed of a resilient material, the material should be flexible enough to allow the height of the raised platform to be adjusted by the vertically adjustable spacers 330. Suitable materials for the base portion and raised platform include acrylic, resin, rubber or plastic such as rigid thermoplastic or polyurethane. Examples of possible thermoplastics include thermoplastic elastic polymers mixed with polycaprolactone and polyvinyl acetate. The thermoplastic elastic polymers may be, for example, ethylene/vinyl acetate copolymers. Other examples of suitable materials are materials commonly used to manufacture dental appliances such as mouth guards. The connectors 315 and vertically adjustable spacers 330 are preferably made of dental grade metals or similar rigid materials.

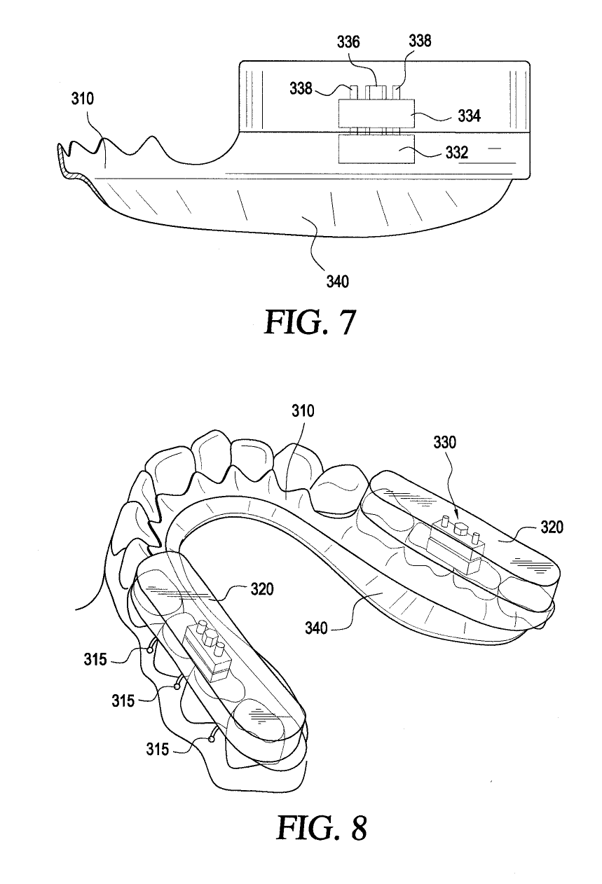

FIG. 7 illustrates a cutaway side view of an exemplary embodiment of the present invention taken at lines X-X of FIG. 3. FIG. 7 illustrates base portion 310 including the raised portion 320 and optional retaining flange 340. This view more clearly shows the vertically adjustable spacer 330 and component parts that adjust the height of the raised platform 320. The vertically adjustable spacer 330 includes an optional lower platform 332 and an upper platform 334 that adjusts the height of the raised platform 320. The vertically adjustable spacer 330 also includes an adjustment screw 336 and guide rods 338 that helps to evenly distribute the force exerted by the vertically adjustable spacer 330 within the interior of raised platform 320. As a result of the exerted force, raised platform 320 stretches or expands thereby exerting a force against the patient's teeth and modifying alignment of the jaw.

FIG. 8 illustrates a rear perspective view of an exemplary embodiment of the present invention. This view more clearly illustrates the position of said raised platforms 320 above the molar teeth in the rear of the mouth. The position of the raised platforms 320 in the rear of the mouth acts to shift and reposition the mandible.

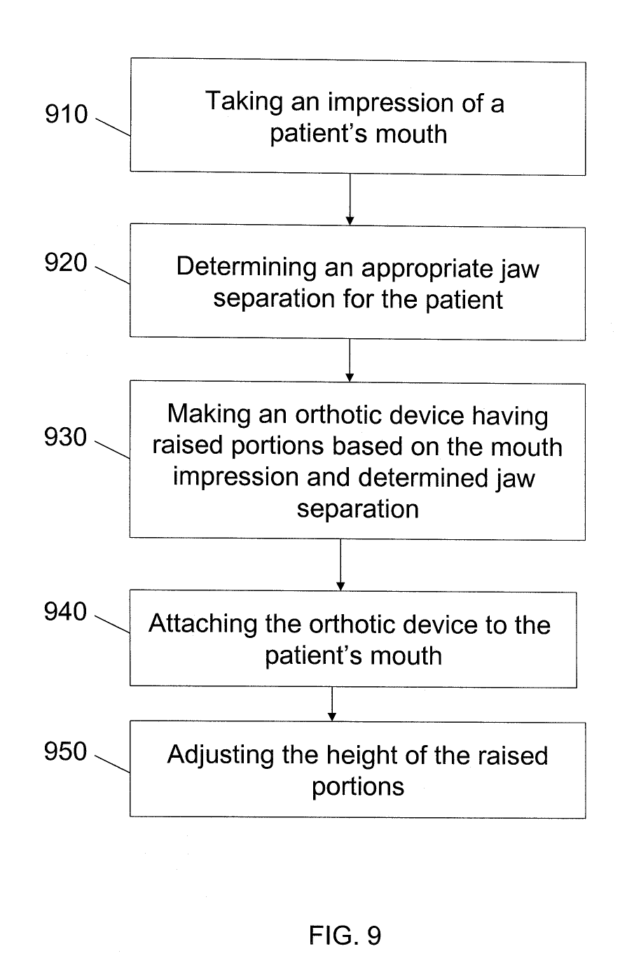

FIG. 9 illustrates the process of adjusting the temporomandibular joint (TMJ) of the present invention. The process of adjusting or realigning the TMJ according to an exemplary embodiment of the present invention begins at 910 by taking an impression of a patient's mouth. The process continues at 920, by determining an appropriate jaw separation between the upper jaw and lower jaw of the patient wherein the appropriate jaw separation is selected to reposition the mandible and relieve disorders associated with misaligned TMJs. At 930, the process continues by making an orthotic device having raised portions based on the mouth impression and determined jaw separation. At 940, the process proceeds to attaching the orthotic device to the patient's mouth. At 950, the process is completed by adjusting/readjusting the height of the raised portions of the orthotic device in order to suitably adjust/realign the TMJ joint.

The present invention may be embodied in a variety of alternative arrangements including, for example, retainer, mouth guard, and bite block type base portions. These base portions may also include a variety of adjustable raised platform arrangements wherein the raised platforms are preferably disposed to be positioned proximate the molar teeth in the rear of the patient's mouth. The exemplary and alternative embodiments described above may be combined in a variety of ways with each other. When there is engagement between two different components, the engagement elements may be reversed between the two components.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

We claim:

1. An apparatus for changing the alignment of the temporomandibular joint, comprising:
an orthotic base that affixes to the interior of a mouth;
at least one raised platform attached to said base; and
at least one adjustable spacer disposed inside said raised platform, wherein said adjustable spacer changes the height of said raised platform.

2. An apparatus according to claim 1, wherein said adjustable spacer includes:
at least one substantially flat platform;
at least one adjustable connection mechanism in communication with said substantially flat platform,
wherein said adjustable connection mechanism is operable to adjust the position of said substantially flat platform, and
wherein said substantially flat platform adjusts the height of said raised platform.

3. An apparatus according to claim 2, wherein said adjustable connection mechanism is exposed.

4. An apparatus according to claim 2, wherein said adjustable spacer further includes at least one guide rod in communication with said at least one substantially flat platform.

5. An apparatus according to claim 1, wherein said device affixes to the mouth by attaching to teeth.

6. An apparatus according to claim 1, wherein said raised platforms are disposed on said base portion to be positioned proximate the molar teeth.

7. An apparatus according to claim 1, further comprising at least one tongue retaining flange attached to said orthotic base.

8. An apparatus for changing the alignment of the temporomandibular joint, comprising:
an orthotic base that affixes to the interior of a mouth;
at least one raised platform attached to said base, wherein said at least one raised platform adjusts the position of the mandible; and
at least one adjustable spacer disposed inside said raised platform, wherein said adjustable spacer changes the height of said raised platform,
said adjustable spacer comprising:
a substantially flat lower platform;
a substantially flat upper platform positioned above said lower platform;
at least one adjustable connection mechanism in communication with said lower bar and said upper bar,
wherein said connection mechanism adjusts the position of said upper platform with respect to said lower platform thereby adjusting the height of said raised platform.

9. An apparatus according to claim 8, wherein said adjustable connection mechanism is exposed.

10. An apparatus according to claim 8, wherein said adjustable spacer further includes at least one guide rod in communication with at least one of said substantially flat platforms.

11. An apparatus according to claim 8, wherein said device affixes to the mouth by attaching to teeth.

12. An apparatus according to claim 8, wherein said at least one raised platform is disposed on said base portion to be positioned proximate the molar teeth.

13. An apparatus according to claim 8, further comprising at least one tongue retaining flange attached to said orthotic base.

14. A method of making a device for changing the alignment of the temporomandibular joint, comprising:
forming an orthotic device base, wherein said base is formed to affix to the interior of a mouth;
attaching at least one raised platform to said base, wherein said at least one raised platform is designed to adjust the position of the mandible; and
disposing at least one adjustable spacer inside said raised platform, wherein said adjustable spacer changes the height of said raised platform.

15. An apparatus according to claim 14, further comprising disposing said adjustable spacer to be exposed.

16. A method according to claim 14, further comprising disposing a connection means on said orthotic base, wherein said connection means affixes to the interior of the mouth.

17. A method for adjusting the position of the mandible, comprising:
taking an impression of a patient's mouth to be adjusted;
determining an appropriate separation of the upper and lower jaw of said mouth;
making an orthotic device having a base and adjustable raised portions, wherein said device integrates said mouth impression and said determined appropriate jaw separation;
attaching said orthotic device to said mouth such that the raised portions are disposed adjacent the patient's molar teeth;
adjusting the height of said raised portions to achieve the desired alignment of the mandible.

18. A method according to claim 17, further comprising utilizing said adjustable spacer to readjust the height of said raised platform.

19. A method according to claim 17, further comprising disposing said raised platforms to be positioned on said base portion proximate the molar teeth.

20. A method according to claim 17, further comprising disposing at least one tongue retaining flange on said orthotic base.
